(19) United States
(12) Patent Application Publication
(10) Pub. No.: US 2016/0243144 A1
(43) Pub. Date: Aug. 25, 2016

(54) FIXED DOSE COMBINATION FORMULATIONS OF RIFAXIMIN AND LACTULOSE

(71) Applicant: Oakdene Holdings LLC, Mendham, NJ (US)

(72) Inventors: James Garegnani, Somerset, NJ (US); Aman Trehan, Somerset, NJ (US)

(73) Assignee: Oakdene Holdings LLC, Mendham, NJ (US)

(21) Appl. No.: 15/049,293

(22) Filed: Feb. 22, 2016

Related U.S. Application Data
(60) Provisional application No. 62/119,121, filed on Feb. 21, 2015.

(51) Int. Cl. A61K 31/7016; A61K 31/437; A61K 9/00

(52) U.S. Cl. CPC A61K 31/7016; A61K 9/0053; A61K 31/437

(57) ABSTRACT

A fixed dose combination formulation of rifaximin or a pharmaceutically acceptable salt thereof and lactulose is provided. The formulation is used for the prevention, treatment, or maintaining remission of HE; reducing the recurrence of HE episodes; reducing the recurrence of HE episodes in a patient refractory or non-responsive to rifaximin or lactulose monotherapy; or reducing the frequency of hospital visit.

### CROSS-REFERENCE TO RELATED APPLICATIONS

This application is a nonprovisional application claiming the benefit priority of U.S. Provisional Application No. 62/119,121, filed Feb. 21, 2015, the contents of which are incorporated herein in their entirety by reference.

### BACKGROUND OF THE INVENTION

(a) Field of the Invention

The present invention is directed to a fixed dose combination formulation of a non-absorbable disaccharide and a gastrointestinal specific antibiotic for prevention, treatment, or maintaining remission of hepatic encephalopathy (HE). More particularly, the invention relates to a fixed dose combination formulation of rifaximin or a pharmaceutically acceptable salt thereof and lactulose and use of such formulation for prevention, treatment, or maintaining remission of HE; reducing the recurrence of HE episodes; reducing the recurrence of HE episodes in a patient refractory or non-responsive to rifaximin or lactulose monotherapy; or reducing the frequency of hospital visits.

(b) Description of the Related Art

Hepatic encephalopathy (HE) is a serious neuropsychiatric complication that is seen in patients with liver dysfunction or failure. The wide spectrum of neuropsychiatric abnormalities of HE range from mentation, neuromuscular abnormalities, mild disturbances in cognitive function and consciousness to coma and death.

HE occurs in 1 of every 3 cases of cirrhosis, in cases of fulminant hepatic failure reported in the United States, and is present in nearly half of patients reaching end-stage liver disease. It may occur at any age, but the peaks parallel those of fulminant liver disease (peak=40's), and cirrhosis (peak=late 50's).

The pathogenesis of HE in cirrhosis is complex and multi-factorial, but a key role is thought to be played by circulating gut-derived toxins of the nitrogenous compounds, most notably ammonia. It is considered as the main culprit behind primary and secondary symptoms making it the center of potential therapeutic options for the treatment of HE. Most treatments of hyperammonemia target the organs and metabolic processes involved in ammonia detoxification. Treatment strategies are directed at increased elimination or reduction of ammonia in addition to correction of dynamic conditions that provoke stints of HE. Overall, a key strategy in the treatment of HE is to target the hyperammonemia by reducing production and absorption of ammonia from the gastrointestinal tract through empirical therapy.

Hepatic encephalopathy is manifested as a continuum of psychomotor dysfunction, impaired memory, increased reaction time, sensory abnormalities, poor concentration and in severe forms, as coma. Changes may be observed in personality, consciousness, behaviour and neuromuscular function. Neurologic signs may include hyperreflexia, rigidity, myoclonus and asterixis (coarse “flapping” muscle tremor). Cognitive tasks such as connecting numbers with lines can be abnormal. Fetor hepaticus (sweet breath odor) may be present. Electroencephalogram (EEG) tracings show nonspecific slow, triphasic wave activity mainly over the frontal areas. Prothrombin time may be prolonged and not correctable with Vitamin K. A computed tomography scan of the head may be normal or show general atrophy. Finally, signs of liver disease such as jaundice and ascites may be noted.

Diagnosis of HE is made on the basis of medical history, and physical and mental status examinations with the required clinical elements being knowledge of existent liver disease, precipitating factor(s), and/or prior history of HE. An EEG may show slow-wave activity, even in mild cases. An elevated serum ammonia level is characteristic but not essential, and correlates poorly with the level of encephalopathy.

Management of patients with chronic HE includes 1) provision of supportive care, 2) identification and removal of precipitating factors, 3) reduction of nitrogenous load from the gut, and 4) assessment of the need for long term therapy. The nitrogenous load from the gut is typically reduced using non-absorbable disaccharide (e.g. lactulose) and/or antibiotics.

Conventional therapy aims to lower the production and absorption of ammonia. Management of HE primarily involves avoidance of precipitating factors, limitation of dietary protein intake, and administration of various ammonia-lowering therapies including non-absorbable disaccharides such as lactulose and lactitol, and the gastrointestinal specific antibiotic rifaximin.

Lactulose is metabolized by the intestinal bacteria of the colon, which leads to reduced faecal pH, then to a laxative effect, and finally to faecal elimination. The reduced faecal pH ionizes ammonia (NH₃) to the ammonium ion (NH₄⁺) which is used by the bacteria for amino acid and protein synthesis. This lowers the serum ammonia levels and improves mental function.

Lactulose is typically used in doses of 30-60 g daily and the dose can be titrated up to 20-40 g TID-QID to affect 2-3 semi-formed bowel movements per day. If lactulose cannot be administered orally or per nasogastric tube, for example to patients with stage 3 and 4 HE, it may be given as a 300 cc (200 g) retention enema.

The non-absorbable disaccharide treatment regimens are, however, complex and over-treatment may result in serious adverse effects such as severe dehydration, hyponatremia and in some cases worsening of HE.

Rifaximin is a nonaminoglycoside, semisynthetic antibiotic derived from rifamycin O. It is a non-systemic, non-absorbed, broad-spectrum, oral antibiotic specific for enteric pathogens of the GI tract. Rifaximin was found to be advantageous in treatment of HE relative to previously used antibiotics; e.g., negligible systemic absorption (<0.4%) regardless of food intake or presence of GI disease and exhibits no plasma accumulation with high or repeat doses. Rifaximin acts locally in the gut to inhibit bacterial RNA synthesis by binding irreversibly to the β-subunit of the DNA-dependent RNA polymerase. Rifaximin has a broad spectrum in vivo activity against aerobic and anaerobic Gram-positive and Gram-negative enteric bacteria, and it decreases production of nitrogenous and other neurotoxic compounds that are believed to be important in pathogenesis of HE. In addition, the mechanism of action of rifaximin in the clinical setting may, at least in part, relate to its ability to modify microbiota-associated metabolic function. The lack of systemic absorption makes rifaximin safe and well tolerated, thus improving patient compliance and reducing side effects.

Rifaximin (INN; see The Merck Index, XIII Ed., 8304) is an antibiotic belonging to the rifamycin class of antibiotics, e.g., a pyrido-imidazo rifamycin. Rifaximin is described in Italian Pat. No. IT1154655 and European Pat. No. EP 0,161,534 B1. The EP patent also discloses a process for rifaximin production using rifamycin 0 as the starting material (The Merck Index, XIII Ed., 8301). U.S. Pat. No. 7,045,620 discloses polymorphic forms of rifaximin. The applications and patents referred to here are incorporated herein by reference in their entirety for all purposes.

Rifaximin is represented by the following formula.

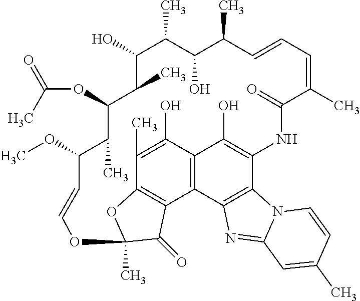

Treatment with oral rifaximin (Refero®, Targaxan®, Tixtellar®, Xifaxan®), a broad-spectrum antibacterial agent that is minimally absorbed and generally well tolerated, and like lactulose, is considered standard care, provides another strategy to reduce ammonia load by reducing enteric bacterial flora, including ammonia-producing bacteria.

Oral rifaximin given 550 mg twice daily, administered either alone or more commonly with medicines containing lactulose, is approved in several countries, including the UK, EU and US, for use in adults with liver disease to reduce the recurrence of episodes of overt HE. From treatment economics, monotherapy of rifaximin is, however, more expensive as compared to the other alternative HE monotherapies such as lactulose, lactitol, neomycin (see Huang E et al., Aliment Pharmacol Ther. 2007 Oct. 15; 26 (8):1147-61).

No single product has been shown to be uniformly effective in the prevention or treatment of HE. Thus, combination therapy is considered as an effective option.

Recently, several clinical studies have concluded that combination therapy of a non-absorbable disaccharide, such as lactulose, and a gastrointestinal specific antibiotic, such as rifaximin, is more effective than disaccharide alone in the treatment of HE.

The efficacy, tolerability and synergistic effect of combined use of rifaximin and lactulose in the treatment of encephalopathy during cirrhosis of the liver have been studied by Puxeddu A. et al. (Current medical research and opinion (1995), 13(5), 274-81). The patients in the study were treated for 15 consecutive days with rifaximin at a dosage of 1200 mg/day in association with sufficient lactulose to induce 2 or 3 evacuations per day. The study concluded that combined use of the two drugs shows good tolerability, efficacy and a synergetic effect in reducing ammonia-producing flora.

In a study by Sharma et al. (American Journal of Gastroenterology (2013), 108(9), 1458-1463), it was concluded that the combination therapy of rifaximin and lactulose may also decrease hospital mortality and hospital stay. The combination therapy group in the study administered one 400 mg rifaximin capsule orally three times a day and 30-60 ml lactulose through a nasogastric tube three times a day.

A publication by Mohammad Rima A. et al. (Annals of Pharmacotherapy (2012), 46(11), 1559-1563, 5 pp) discloses efficacy and safety of various combination therapies for the treatment and prevention of HE. The publication, referring to the study by Puxeddu A et al., suggests that a combination of rifaximin and lactulose may be considered in the treatment of HE and in patients refractory to monotherapy.

Gluud Lise Lotte et al. (Metabolic Brain Disease (2013), 28(2), 221-225) discloses a meta-analysis of clinical studies with an objective of finding evidence for the effect of non-absorbable disaccharides (lactulose and lactitol), rifaximin and branched chain amino acids (BCAA) in HE therapy. The analysis showed that combination of lactulose with rifaximin is more beneficial in HE than lactulose alone.

Mantry P. S. et al. (Transplantation Proceedings (2010), 42(10), 4543-4547) discloses a study performed to evaluate the efficacy and safety of rifaximin in addition to lactulose in improving hospitalization outcomes in patients with HE. The patients in the study received lactulose (20-120 gm/day; lactulose phase) before treatment with rifaximin (400-1200 mg/d; rifaximin phase).

A publication by Leise Michael D. et al. (Mayo Clinic proceedings (2014), 89(2), 241-53) suggests that adding rifaximin to lactulose therapy can benefit patients with recurrent HE episodes. The publication further discloses that if rifaximin is added to lactulose, episodes of overt HE and hospitalization can be prevented compared with lactulose alone.

PCT Application Pub. No. WO 2005065429 discloses a treatment for HE characterized by hyperammonemia and/or constipation by orally administering a composition comprising lactulose in combination with polyethylene glycol.

U.S. Pat. No. 8,642,573 discloses a method of treating HE by administering the GI specific antibiotic, rifaximin with lactulose. The patent further discloses that the patient can administer two 500 mg tablets of rifaximin twice-daily with lactulose. Lactulose use can be titrated over time so that the subject maintains 2-3 soft stool bowl movements per day. Lactulose can be administered in a 15 ml dosage form containing 10 mg of lactulose.

Italian Patent Application No. ITSA20060001 discloses an enteroclysis solution comprising a non-absorbable disaccharide (lactulose or lactitol) and the antibiotic rifaximin for treating HE. A single 800-1200 ml dose of the solution may include 100-300 gm of disaccharide and 0.05-3 gm of rifaximin.

The incidence of HE is likely to increase with the incidence of hepatitis C in the general population and cirrhotics in aging patients. Acute HE signifies a serious prognosis with a 40% likelihood of survival for one year. There is a need for improved therapy options and methods for treating and preventing HE.

Overall, therapeutic treatment options for HE are currently limited to administration of a gastrointestinal specific antibiotic, rifaximin; a non-absorbable disaccharide, lactulose; and simultaneous or co-administration of the two agents in separate dosage forms and/or multiple dosing frequency. The efficacy of treating HE by co-administering rifaximin and lactulose is dependent, at least in part, on the quantity that is administered. For example, administration of a dosage of rifaximin and lactulose will not effectively alleviate HE if the quantity administered is too low. The quantity of rifaximin and lactulose, if higher than the therapeutically essential dose, also plays a role in the occurrence of side effects of each of rifaximin and lactulose, such as peripheral edema (swelling, usually in the ankles or lower limbs), nausea (feeling sick to your stomach), dizziness, fatigue (feeling tired), ascites (a buildup of fluid in the abdomen) in case of rifaximin and dehydration, electrolyte disturbances, abdominal cramping in case of lactulose.

Therefore, it is essential to determine an effective dosing regimen for co-administering rifaximin and lactulose in order to effectively treat HE. An improvement to simplify treatment regimens for HE and other related conditions affected by rifaximin and lactulose would also enhance patient compliance by providing a simplified dosage form containing pharmaceutically acceptable amounts of the two treatments. Such formulation can also avail a viable, cost-effective and economic treatment option to patients in need of HE treatment. There is an unmet need for providing a single dosage form comprising a combination of rifaximin and lactulose in which the active agents are provided in the formulation in quantities sufficient to provide the desired bioavailability and/or desired therapeutic and/or pharmacological response.

### SUMMARY OF THE INVENTION

The present invention generally relates to prevention, treatment, or maintenance of remission of HE or related conditions using a fixed dose combination formulation of a gastrointestinal specific antibiotic and a non-absorbable disaccharide. The fixed dose combination formulation comprises rifaximin or a pharmaceutically acceptable salt thereof and lactulose, optionally with pharmaceutically acceptable excipients.

In a first aspect, the invention provides a fixed dose combination formulation for treating, preventing, or maintaining remission of HE comprising rifaximin or a pharmaceutically acceptable salt thereof, lactulose, and optionally one or more pharmaceutically acceptable excipients.

In a further aspect, the invention provides an oral suspension for treating, preventing, or maintaining remission of HE comprising a fixed dose combination of rifaximin or a pharmaceutically acceptable salt thereof, lactulose, and optionally one or more pharmaceutically acceptable excipients.

In a further aspect, the invention provides a sachet for treating, preventing, or maintaining remission of HE comprising a fixed dose combination of rifaximin or a pharmaceutically acceptable salt thereof, lactulose, and optionally one or more pharmaceutically acceptable excipients.

In a further aspect, the invention provides a multilayer tablet for treating, preventing, or maintaining remission of HE comprising a fixed dose combination of rifaximin or a pharmaceutically acceptable salt thereof, lactulose, and optionally one or more pharmaceutically acceptable excipients.

In a further aspect, the invention provides a capsule for treating, preventing, or maintaining remission of HE comprising a fixed dose combination of rifaximin or a pharmaceutically acceptable salt thereof, lactulose, and optionally one or more pharmaceutically acceptable excipients.

In a further aspect, the daily dose of rifaximin or a pharmaceutically acceptable salt thereof in the fixed dose combination formulation is about 25 mg to about 6000 mg, and preferably about 500 mg to about 1500 mg.

In a further aspect, the daily dose of lactulose or a pharmaceutically acceptable salt thereof in the fixed dose combination formulation is about 10 mg to about 20 gm, and preferably about 10 mg to about 2000 mg.

In a further aspect, the dose of rifaximin or a pharmaceutically acceptable salt thereof in the fixed dose combination formulation is greater than or equal to the dose of lactulose.

In a further aspect, the combined weight of rifaximin and lactulose in the fixed dose combination formulation is less than about 80% of the weight of rifaximin or lactulose alone required to achieve the same therapeutic effect in a patient to which the formulation is administered.

In a further aspect, the ratio of amount of rifaximin or a pharmaceutically acceptable salt thereof to lactulose in the fixed dose combination formulation ranges from about 0.05:1 to about 50:1.

In a further aspect, the ratio of amount of rifaximin or a pharmaceutically acceptable salt thereof to lactulose in the fixed dose combination formulation ranges from about 1:1 to about 50:1.

In a further aspect, the fixed dose combination formulation comprises from about 30% w/w to about 70% w/w of rifaximin or a pharmaceutically acceptable salt thereof.

In a further aspect, the fixed dose combination formulation comprises from about 20% w/w to about 80% w/w of lactulose.

In a further aspect, the invention provides a solid oral fixed dose combination formulation for treating, preventing or maintaining remission of HE comprising rifaximin or a pharmaceutically acceptable salt thereof, lactulose, and optionally one or more pharmaceutically acceptable excipients.

In a further aspect, the invention provides a solid oral fixed dose combination formulation for treating, preventing or maintaining remission of HE comprising rifaximin or a pharmaceutically acceptable salt thereof and lactulose, wherein each active agent in the formulation forms a discrete layer.

In a further aspect, the invention provides a method of treating, preventing, or maintaining remission of HE in a patient in need thereof comprising administering a fixed dose combination formulation comprising rifaximin or a pharmaceutically acceptable salt thereof and lactulose.

In a further aspect, the fixed dose combination formulation comprising rifaximin or a pharmaceutically acceptable salt thereof and lactulose is administered for greater than 365 days, greater than 730 days, greater than 1095 days, or for the remainder of the life of the patient having HE.

In a further aspect, the invention provides a fixed dose combination formulation lactulose for treating, preventing or maintaining remission of HE comprising rifaximin or a pharmaceutically acceptable salt thereof and, wherein once-a-day administration of said formulation provides steady state blood levels of rifaximin or lactulose that are comparable to steady state blood levels of rifaximin or lactulose achieved with simultaneous administration of separate dosage forms containing the same amounts of rifaximin and lactulose.

In a further aspect, once-a-day administration of the fixed dose combination formulation provides steady state blood Cₘₐₓ levels of rifaximin in the range of about 17 to 35 ng/ml, AUC in the range of 110 to 250 ng-hr/ml and Tmax with a median of approximately 1 hour. It is reported that lactulose given orally to man and experimental animals resulted in only small amounts reaching the blood. Urinary excretion has been determined to be 3% or less and is essentially complete within 24 hours. Therefore in this aspect the Cₘₐₓ and AUC levels of lactulose are very low and variable.

In a further aspect, the invention provides a method of reducing at least one side effect of rifaximin and/or lactulose therapy in patient suffering from HE, said side effects being selected from peripheral edema, nausea, dizziness, fatigue, ascites, dehydration, electrolyte disturbances, abdominal cramping, and combinations of said side effects, comprising administering to said patient a fixed dose combination formulation comprising rifaximin or a pharmaceutically acceptable salt thereof and lactulose.

In a further aspect, the invention provides a method of reducing the frequency of hospitalization visits by a patient suffering from HE, comprising administering to the patient a fixed dose combination formulation comprising rifaximin or a pharmaceutically acceptable salt thereof and lactulose.

In a further aspect, the invention provides a method of increasing the length of time to hospitalization readmission for a patient suffering from HE, comprising administering to the patient a fixed dose combination formulation comprising rifaximin or a pharmaceutically acceptable salt thereof and lactulose.

In a further aspect, the invention provides a method of reducing the recurrence of HE episodes in a patient comprising administering to the patient a fixed dose combination formulation comprising rifaximin or a pharmaceutically acceptable salt thereof and lactulose.

In a further aspect, the invention provides a method of reducing the recurrence of HE episodes in a patient refractory, intolerant, or non-responsive to rifaximin or lactulose monotherapy comprising administering to the patient a fixed dose combination formulation comprising rifaximin or a pharmaceutically acceptable salt thereof and lactulose.

Still other aspects and advantages of the invention will be apparent from the following

### DETAILED DESCRIPTION OF THE INVENTION

The invention addresses the need for effective prevention, treatment, or maintenance of remission of HE and related conditions by providing a fixed dose combination formulation of rifaximin or a pharmaceutically acceptable salt thereof and lactulose.

In general, the fixed dose combination of the active agents may show unpredictable problems attributed to pharmacokinetic, therapeutic or pharmaceutical properties of the individual active agent formulated into a single dosage form. Rifaximin is low in both solubility and bioabsorption (BCS Class IV, low solubility, low permeability), and varies greatly in bioavailability even when it is administered in a single dosage form. Further, rifaximin is known to be a highly variable drug (exhibiting intra-subject variability of 30% CV or greater in Cₘₐₓ or AUC), particularly in patients with HE. Lactulose, in contrast, is known to have good solubility and a poor gastrointestinal absorption rate. The difficulties in formulating a viable and effective combination dosage form of the two active agents thus may not be envisaged considering said differences between the two active ingredients. Another problem with the formulation of a fixed dose combination formulation of the two active ingredients is the unpredictability of likelihood of stability issues due to interaction between the different active ingredients and/or pharmaceutically acceptable excipients.

Hence, it is beyond the ordinary skill in the art and challenging to develop a fixed dose combination formulation of rifaximin and lactulose.

In addition, due to the high dose of rifaximin or a pharmaceutically acceptable salt thereof (550 mg of the free base per tablet) and lactulose (up to 40 gm of daily dose) currently required for effective treatment of HE, it is not trivial to make the combination formulation of the two active agents that is suitable for the desired therapeutic purpose.

The inventors have unexpectedly found that the fixed dose combination formulation of rifaximin and lactulose achieves at least the same or greater biological, pharmacological, or therapeutic effect relative to separate dosage forms and/or multiple daily dose therapy of the two active agents as suggested in the prior art. Particularly, it was found that a fixed dose formulation comprising rifaximin and lactulose in a specific dose proportion improves the symptoms of HE or related conditions and improves compliance to the treatment as compared to the currently known therapy regimens. A further advantage of the fixed dose formulation of the inventions is reduction in the daily dose of individual active agent and/or frequency of administration.

For a fixed dose combination formulation of the present invention, the side effects can also be minimized compared to conventional individual single or combined dosage forms. Also the formulation can exhibit higher stability over the labelled storage period.

Accordingly, embodiments of the invention are directed to a fixed dose combination formulation for prevention, treatment, or maintenance of remission of HE. The formulation comprises rifaximin or a pharmaceutically acceptable salt thereof, lactulose and optionally with one or more pharmaceutically acceptable excipients. Accordingly, the fixed dose combination formulation of the present invention can be used to prevent, treat, or maintain remission of HE.

Embodiments are also directed to a solid oral fixed dose combination formulation comprising rifaximin or a pharmaceutically acceptable salt thereof and lactulose.

Embodiments are also directed to a fixed dose combination formulation in the form of an oral suspension, tablet, multilayer tablet, sachet or capsule comprising rifaximin or a pharmaceutically acceptable salt thereof and lactulose.

Embodiments are also directed to a solid oral fixed dose combination formulation comprising rifaximin or a pharmaceutically acceptable salt thereof and lactulose, wherein each active ingredient in the formulation forms a discrete layer.

Embodiments are also directed to a fixed dose combination formulation comprising about 25 mg to about 6000 mg daily dose of rifaximin or a pharmaceutically acceptable salt thereof, and preferably from about 500 mg to about 1500 mg daily dose of rifaximin or a pharmaceutically acceptable salt thereof.

Embodiments are also directed to a fixed dose combination formulation comprising about 10 mg to about 20 gm daily dose of lactulose, and preferably from about 10 mg to about 2000 mg daily dose of lactulose. In an embodiment, the daily dose of lactulose in the fixed dose combination formulation is from about 500 mg to about 2000 mg.

Embodiments are also directed to a fixed dose combination formulation comprising rifaximin or a pharmaceutically acceptable salt thereof and lactulose, wherein the dose of rifaximin or a pharmaceutically acceptable salt thereof is greater than or equal to the dose of lactulose.

Embodiments are also directed to a fixed dose combination formulation, wherein the combined weight of rifaximin and lactulose in the formulation is less than about 80% of the weight of rifaximin or lactulose alone required to achieve same therapeutic effect in a patient to which the formulation is administered.

Embodiments are also directed to a fixed dose combination formulation comprising rifaximin or a pharmaceutically acceptable salt thereof and lactulose, wherein the ratio of amount of rifaximin or a pharmaceutically acceptable salt thereof to lactulose ranges from about 0.05:1 to about 50:1.

Embodiments are also directed to a fixed dose combination formulation comprising rifaximin or a pharmaceutically acceptable salt thereof and lactulose, wherein the ratio of amount of rifaximin or a pharmaceutically acceptable salt thereof to lactulose ranges from about 1:1 to about 50:1.

Embodiments are also directed to a fixed dose combination formulation comprising rifaximin or a pharmaceutically acceptable salt thereof and lactulose, wherein the formulation comprises from about 30% w/w to about 70% w/w of rifaximin or a pharmaceutically acceptable salt thereof.

Embodiments are also directed to a fixed dose combination formulation comprising rifaximin or a pharmaceutically acceptable salt thereof and lactulose, wherein the formulation comprises from about 20% w/w to about 80% w/w of lactulose.

Embodiments are also directed to a fixed dose combination formulation comprising rifaximin or a pharmaceutically acceptable salt thereof and lactulose for treating, preventing or maintaining remission of HE, wherein once-a-day administration of said formulation provides steady state blood levels of rifaximin or lactulose that are comparable to steady state blood levels of rifaximin or lactulose achieved with simultaneous administration of separate dosage forms containing same amount of rifaximin and lactulose. In an embodiment, once-a-day administration of the fixed dose combination formulation provides steady state blood Cₘₐₓ levels of rifaximin in the range of about 17 to 35 ng/ml, AUC in the range of 110 to 250 ng-hr/ml and Tmax with a median of approximately 1 hour.

Embodiments are also directed to a method of reducing at least one side effect of rifaximin and/or lactulose therapy in patient suffering from HE, said side effects being selected from peripheral edema, nausea, dizziness, fatigue, ascites, dehydration, electrolyte disturbances, abdominal cramping, and combinations of said side effects, comprising administering to said patient a fixed dose combination formulation comprising rifaximin or a pharmaceutically acceptable salt thereof and lactulose.

Embodiments are also directed to a method of treating, preventing, or maintaining remission of HE in a patient in need thereof comprising administering a fixed dose combination formulation comprising rifaximin or a pharmaceutically acceptable salt thereof and lactulose.

Alternatively, the method of treating, preventing, or maintaining remission of HE in a patient in need thereof may comprise administering the fixed dose combination formulation comprising rifaximin or a pharmaceutically acceptable salt thereof and lactulose, and restricting the patient's diet to non-protein containing food.

Embodiments are also directed to a method of reducing the frequency of hospitalization visit by a patient suffering from HE comprising administering to the patient a fixed dose combination formulation comprising rifaximin or a pharmaceutically acceptable salt thereof and lactulose. In an embodiment, treatment with the fixed dose combination formulation of the invention reduces hospitalization frequency by between about 10% to about 70%.

Embodiments are also directed to a method of reducing the recurrence of HE episodes in a patient comprising administering to the patient a fixed dose combination formulation comprising rifaximin or a pharmaceutically acceptable salt thereof and lactulose.

Embodiments are also directed to methods of reducing the recurrence of HE episodes in a patient refractory or non-responsive to rifaximin or lactulose monotherapy comprising administering to the patient a fixed dose combination formulation comprising rifaximin or a pharmaceutically acceptable salt thereof and lactulose.

The term “fixed dose combination formulation” refers to a formulation of two or more medications or active ingredients combined in a single unit dosage form, and available in certain fixed doses.

The term “administration” or “administering” includes routes of introducing a fixed dose combination of rifaximin and lactulose to a patient to perform their intended function. Examples of routes of administration that may be used include injection (subcutaneous, intravenous, parenterally, intraperitoneally, intrathecal), oral, inhalation, rectal and transdermal.

The term “rifaximin” as used herein is meant to cover solvates and crystalline and amorphous forms of rifaximin in the form of free base or its pharmaceutically acceptable salt(s), hydrate(s), and physiologically functional derivative(s) and precursors thereof. The formulation of the invention may contain rifaximin alone or in a mixture with other polymorphic forms of rifaximin to modulate the in vivo absorption.

The term “prevention” as used herein relates to treatment in order to prevent the development of a disease or a condition. The preventive treatment is normally used on individuals who have not yet shown any clinical signs of HE or related conditions.

The term “treatment” used herein relates to treatment in order to cure or alleviate a disease or a condition. The treatment may either be performed in an acute or in a chronic manner.

As used herein, the term “remission” means diminution or abatement of the symptoms of a disease or a condition; the period during which such diminution occurs.

As used herein, the term “recurrence” means the return of a disease or a condition after a remission.

The term “pharmaceutically acceptable salts” as used herein refers to salts which are known to be non-toxic and are commonly used in the pharmaceutical literature. Typical inorganic acids used to form such salts include hydrochloric, hydrobromic, hydroiodic, nitric, sulfuric, phosphoric, hypophosphoric, and the like. Salts derived from organic acids, such as aliphatic mono and dicarboxylic acids, phenyl substituted alkanoic acids, hydroxyalkanoic and hydroxyalkandioic acids, aromatic acids, aliphatic and aromatic sulfonic acids, may also be used. Such pharmaceutically acceptable salts thus include acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, beta-hydroxybutyrate, chloride, cinnamate, citrate, formate, fumarate, glycolate, heptanoate, lactate, maleate, hydroxymaleate, malonate, mesylate, nitrate, oxalate, phthalate, phosphate, monohydro genphosphate, dihydrogenphosphate, metaphosphate, pyrophosphate, propionate, phenylpropionate, salicylate, succinate, sulfate, bisulfate, pyrosulfate, sulfite, bisulfite, sulfonate, benzenesulfonate, p-bromophenylsulfonate, chlorobenzenesulfonate, ethanesulfonate, 2-hydroxyethanesulfonate, methanesulfonate, naphthalene-1-sulfonate, naphthalene-2-sulfonate, p-toluenesulfonate, xylenesulfonate, tartarate, and the like.

The abbreviations used herein generally have their conventional meaning within the chemical and biological arts.

“Rifaximin”, as used herein, includes solvates and polymorphous forms of the molecule, including, for example, Form α, Form β, Form γ Form δ, Form ε, Form ζ, Form η, Form i, Form kappa, Form theta, Form mu, Form omicron, Form pi, mesylate Form or amorphous forms of rifaximin. These forms are described in more detail, for example, in U.S. Pat. No. 7,045,620; U.S. Pat. No. 7,612,199; U.S. Pat. No. 7,709,634; U.S. Pat. No. 7,915,275; U.S. Pat. No. 8,067,429; U.S. Pat. No. 8,193,196; U.S. Pat. No. 8,227,482; U.S. Pat. No. 8,383,151; U.S. Pat. No. 8,486,956; U.S. Pat. No. 8,513,275; U.S. Pat. No. 8,518,949; G. C. Viscomi, et al., Cryst. Eng. Comm, 2008, 10, 1074-1081 (April 2008), and US Pat. Application Publication No. 2005/0272754. Each of these references is hereby incorporated by reference in entirety.

Rifaximin exerts its broad antibacterial activity, for example, in the gastrointestinal tract against localized gastrointestinal bacteria that cause infectious diarrhea, irritable bowel syndrome, small intestinal bacterial overgrowth, Crohn's disease, and/or pancreatic insufficiency. It has been reported that rifaximin is characterized by a negligible systemic absorption, due to its chemical and physical characteristics (Descombe J. J. et al. Pharmacokinetic study of rifaximin after oral administration in healthy volunteers. Int J Clin Pharmacol Res, 14 (2), 51-56, (1994)).

Without wishing to be bound by any particular theory, rifaximin acts by binding to the beta-subunit of the bacterial deoxyribonucleic acid-dependent ribonucleic acid (RNA) polymerase, resulting in inhibition of bacterial RNA synthesis. It is active against numerous gram (+) and (−) bacteria, both aerobic and anaerobic. In vitro data indicate rifaximin is active against species of Staphylococcus, Streptococcus, Enterococcus, and Enterobacteriaceae. Bacterial reduction or an increase in antimicrobial resistance in the colonic flora does not frequently occur and does not have a clinical importance. Rifaximin is currently approved in 17 countries outside the US and was licensed by the Food and Drug Administration (FDA) for the US in May 2004.

Rifaximin for the purpose of the present invention may be prepared by spray drying and characterized by a substantially symmetric profile of the particle size distribution around 20 μm particles diameter, wherein the measure of d90 (percentage of 90% of the particles diameter) is between 40 and 120 μm, d50 (percentage of 50% of the particles diameter) is between 15 and 30 μm, and d10 (percentage of 10% of the particles diameter) is between 2 and 10 μm. Rifaximin obtained by a milling process is characterized by a non-symmetric profile, wherein the d90 is between 10 and 20 μm, d50 is between 6 and 12 μm, and d10 is between 0.5 and 4 μm.

The bulk density of rifaximin prepared by spray drying is between 0.1 and 0.5 g/ml and the density of rifaximin by milling is between 0.3 and 0.6 g/ml.

The specific surface area (BET) of rifaximin prepared by milling can range from 5 to 20 m²/g, and more particularly between 9 to 12 m²/g, while the BET of rifaximin by spray drying is between 0.01 to 10 m²/g and more particularly between 5 to 8 m²/g.

Lactulose for the practice of the invention is readily available as an over-the-counter product. A convenient and relatively tasteless formulation, often referred to in the trade as “lactulose powder for oral solution” can be obtained, for example, from Bertek Pharmaceuticals, Sugarland, Tex. as Kristalose® in 10 and 20 g packets.

Additionally, one or more additional active agents suitable for prevention or treatment of HE may be combined with the fixed dose combination formulation of the invention for effective management of HE. Such additional active agents include, but not limited to lactitol, neomycin, metronidazole, L-ornithine, and L-aspartate, vancomycin, Bifidobacterium infantis 35624, Nitazoxanide, Mesalamine, Cholestyramine, Sandostatin, Lactose, Lubiprostone, Omeprazole, Pantoprazole, Sodium bicarbonate, and Polyethylene Glycol 3350.

A fixed dose combination formulation of the invention is formulated to be compatible with its intended route of administration. For example, these preparations are administered in solution, suspension, tablets, multilayer tablets, powder or granules in sealed packet/sachet or hard and soft gelatine capsule, by injection or infusion, inhalation, eye lotion, eye drops, ointment, cream, gel, lotion, suppository etc. Oral administration is preferred.

Oral formulations generally include an inert diluent or an edible carrier. Suitable oral formulations may be, e.g., enclosed in gelatin capsules or compressed into tablets. For the purpose of oral therapeutic administration, the active agents can be incorporated with excipients and used in the form of tablets, troches, capsules, lozenges (using a flavored basis, usually sucrose and acacia or tragacanth), powders, granules, or as a solution or a suspension in an aqueous or nonaqueous liquid, or as an oil-in-water or water-in-oil liquid emulsion, or as an elixir or syrup, or as pastilles (using an inert base, such as gelatin and glycerin, or sucrose and acacia), each containing a predetermined amount of rifaximin and lactulose. Pharmaceutically compatible binding agents, and/or adjuvant materials can be included as part of the formulation. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

It is especially advantageous to formulate oral formulations in dosage unit form for ease of administration and uniformity of dosage. The term “dosage unit form” as used herein refers to physically discrete units suited as unitary dosages for the patient to be treated; each unit containing a predetermined quantity of active agent(s) calculated to produce the desired therapeutic effect in association with the required pharmaceutical carrier. The specification for the dosage unit forms of the invention are dictated by and directly dependent on the unique characteristics of the active agents and the particular therapeutic effect to be achieved, and the limitations inherent in the art of compounding such active agents for the treatment of individuals.

In an embodiment, the fixed dose formulations for oral administration is administered in immediate release dosage forms.

In a further embodiment, the fixed dose formulations for oral administration can also be administered in controlled release dosage forms. For example, controlled release dosage forms as described hereinafter may be administered every 6, 12 or 24 hours comprising either the same or up to a several fold increase of the amount of active agent(s) in immediate-release dosage form.

Controlled-release of the rifaximin, or pharmaceutically acceptable salts thereof and optionally lactulose, may be affected by incorporating the rifaximin, or pharmaceutically acceptable salts thereof or lactulose, into, by way of example and not limitation, hydrophobic polymers, including acrylic resins, waxes, higher aliphatic alcohols, polylactic and polyglycolic acids and certain cellulose derivatives, such as hydroxypropylmethyl cellulose. In addition, the controlled release may be affected by using other polymer matrices, liposomes and/or microspheres. The controlled release formulation of rifaximin and lactulose may be released at a slower rate and over a longer period of time. For example, in some embodiments, the controlled release formulation of rifaximin and lactulose may release effective amounts of a mixture of rifaximin and lactulose over 12 hours. In other embodiments, the controlled release formulation may release effective amounts of rifaximin and lactulose over 4 hours or over 8 hours. In still other embodiments, the controlled release formulation may release effective amounts of rifaximin and lactulose over 15, 18, 24 or 30 hours.

In some embodiments, the fixed dose combination formulation is administered in an immediate release oral dosage form or, alternatively, is administered in a controlled release oral dosage form. In an embodiment, the rifaximin part of the fixed dose combination formulation exhibits controlled release and the lactulose part exhibits immediate release.

In an embodiment, the fixed dose combination formulation is in the form of a bilayer, a trilayer or a multilayer tablet. A bilayer tablet can be formed by compressing together a first blend comprising rifaximin and a separate, second blend comprising lactulose. Such formulation comprises discrete layers of rifaximin and lactulose. A trilayer tablet can be formed by compressing one or two parts of a blend of either of rifaximin or lactulose together. A trilayer tablet may comprise two layers of rifaximin with a middle layer of lactulose or two layers of lactulose with a middle layer of rifaximin. Alternatively, a trilayer tablet can be formed by compressing a separating or intermediate layer in between the rifaximin layer and lactulose layer. A multilayer tablet can be formed by compressing together single or a plurality of blends of rifaximin, lactulose, and optionally, of pharmaceutically acceptable excipients.

In a further embodiment, the fixed dose combination formulation is in the form of a coated unit dosage form. The coated dosage form may in the form of a core comprising rifaximin and/or lactulose and one or more coating layers surrounding the core, which may also include rifaximin, and/or lactulose, and optionally, pharmaceutically acceptable excipients. The methods and techniques for preparing the core and coating are well known in the art.

A layer may be configured in various ways. For example, in some embodiments a layer comprises a flat portion of a formulation. In some embodiments a layer comprises a rounded portion of a formulation. In some embodiments a layer comprises a conical section of a formulation. In some embodiments a layer comprises an elliptical section of a formulation. In some embodiments a layer comprises a sideways section of a formulation. In some embodiments a layer comprises a cubical section of a formulation. In some embodiments a layer comprises a wedge of a formulation. In some embodiments a layer comprises a substantial portion of a formulation. A substantial portion of the formulation is preferably at least about 25% of the formulation and more preferably at least about 50% of the formulation.

In an embodiment, the fixed dose combination formulation comprises an intermediate layer between rifaximin and lactulose layers, wherein the intermediate layer dissolves rapidly to release discrete parts of rifaximin and lactulose upon contact with body fluids after administration.

In an embodiment, the fixed dose combination formulation may comprise rifaximin and lactulose finely and homogenously dispersed in one or more polymeric carriers. Such mixture is described in the art as solid dispersions, glass solutions, molecular dispersions, and solid solutions. Such dosage form can be prepared by processing the mixture of active agents through the hot-melt extrusion process in which amorphous forms of a crystalline active compound are formed in-situ and dispersed within the polymer matrix.

In a preferred embodiment, the fixed dose combination formulation comprising rifaximin or a pharmaceutically acceptable salt thereof is in the form of an oral suspension.

The oral suspension dosage form may comprise a taste masking agent that is capable of masking the taste of the active agent and also provides the active agent in a suitable form to dissolve thereby providing patient compliance, especially for children and the elderly.

The term ‘taste masking’ as used herein is meant to refer to a reduction of a perceptible unpleasant taste associated with rifaximin in the formulation and/or after stability. The term ‘stable’ as used in the present invention relates to both chemical (shelf-life) and physical stability (suspension uniformity). Improved uniformity results in an improved product because less shaking of the suspension is required before dosing and allows the product to be stored longer (i.e. longer shelf-life) because the drug in the product will not settle and compact.

For the purposes of the oral suspension or any other dosage in accordance with the present invention, rifaximin and optionally lactulose are milled or micronized in the suspension. In order to produce a suspension having the desired particle size, the suspension is milled by various techniques such as, for example, conventional comminution and de-agglomeration, grinding in an air-jet mill or impact mill, a ball mill, dry milling, wet milling, colloidal mill, and dyano mill; and micronization and microfluidization techniques may be used. Using the above techniques helps in obtaining the desired particle size with increased wettability, solubility and dissolution of rifaximin. Small particle size is desirable for reasons other than slowing the rate of sedimentation. For drugs that are not very soluble, smaller particles generally dissolve faster due to the increase in the total surface area. Also, smaller drug particles are less likely to cause grittiness, which improves the palatability of the finished product. There is therefore a need for a suspension containing fine particles, hereafter referring to particles having a d90 less than about 50 μm, which will not cake on storage, but in addition is able to maintain its homogeneity on prolonged storage.

The term ‘d90 less than about 50 μm’ as used herein means that “90% particles are less than about 50 μm”. It is noted that the notation dx means that X % of particles have a diameter less than the specified diameter d.

The d90 of the rifaximin dispersed or suspended in the suspension is less than about 50 μm and more preferably of less than about 30 μm. The particle size of the rifaximin particles can be measured using light scattering techniques (e.g., Malvern Mastersizer Hydro 2000S).

Another important property of the suspension is viscosity which further plays a role in the stability of suspension by slowing the sedimentation rate of the suspension. It is desired that in the present invention, the viscosity of the suspension should not be so high that pumping and handling would be difficult in industrial practice, but high enough to confer upon the suspension a suitable stability to settling of suspended particles for a reasonable period of time. The viscosity of the suspension should be such that it provides a pourable consistency to the suspension. Preferably, the suspension of the present invention has a viscosity in the range of about 20 cps to about 200 cps when measured by a Brookfield viscometer at room temperature and 100 RPM using spindle no. 2.

The suspension contains a pharmaceutically acceptable suspension base with the active ingredient dispersed in the suspension base. The pharmaceutically acceptable suspension base may be, for example, an aqueous solvent such as water, with the suspending/viscosity enhancing agent dispersed throughout. The pharmaceutically acceptable suspension base may contain various additive(s) which are known to a skilled person in art. The suspension of rifaximin or a pharmaceutically acceptable salt(s) and lactulose according to present invention further comprises at least one or more other pharmaceutically acceptable additive(s) selected from group comprising but not limited to flavouring agent(s), sweetening agent(s), buffering agent(s), preservative(s), suspending agent(s), antioxidant(s), wetting agent(s), dispersing agent(s), pH stabilizing agent(s), taste enhancing agent(s), antifoaming agent(s) and mixtures thereof.

Rifaximin is known to have an unpleasant taste. By a suitable combination of sweetener and flavouring agents the unpleasant taste of the rifaximin can be improved.

A flavoring agent(s) used in the suspension is meant to impart a pleasant flavor and/or odor to a formulation. Suitable flavoring agents include but are not limited to natural and artificial flavors, such as synthetic flavor oils and flavoring aromatics and/or natural oils, extracts from plants, leaves, flowers, fruits and so forth and combinations thereof. Representative suitable flavoring agents may be for example, without limitation, menthol, cinnamon, wintergreen, clove, bay, anise, eucalyptus, thyme, cedar leave, nutmeg, sage, bitter almonds and cassia, vanilla, artificial vanilla, chocolate, artificial chocolate, bubble gum, both natural and artificial fruit flavors, such as cherry flavor, grape flavor, orange flavor, banana flavor, strawberry flavor, lemon flavor, grapefruit flavor and “mint” flavors such as peppermint flavor and spearmint flavor, lime flavor, apple flavor, pear flavor, peach flavor, raspberry flavor, plum flavor, pineapple flavor, apricot flavor and so forth, including combinations of two or more thereof. Flavoring agents are generally provided as a minor component of the formulation in amounts effective to provide a palatable flavor to the formulation. The amount of flavoring agent may depend on a number of factors, including the desired organoleptic effect. The precise amount of sweetening and/or flavoring agent(s) depends on the properties of the agent(s) used, however, generally in an amount that is sufficient to mask the unpleasant taste and/or odor associated with rifaximin as determinable by one skilled in the art. Generally, flavoring agents are present in the formulation in a pharmaceutically acceptable range.

Sweeteners suitable for inclusion in the suspension may be determined by one skilled in the art including, for example, without limitation, both natural and artificial sweeteners such as the representative sweetening agents of intense sweeteners such as sorbitol, sucrose, saccharins such as sodium saccharin, cyclamates such as sodium cyclamates, aspartame, sucralose, thaumatin, acesulfam K, and the like, and sugars such as monosaccharides, disaccharides and polysaccharides. Representative sugars useful in the present invention include, without limitation, xylose, ribose, glucose, mannose, galactose, fructose, dextrose, sucrose, maltose, partially hydrolyzed starch or corn syrup, and sugar alcohols such as sorbitol, xylitol, mannitol, glycerin, etc. and combinations thereof. Presently preferred as a sugar sweetener is sucralose. Sugar sweeteners may be replaced or augmented by water-soluble artificial sweeteners, such as the suitable artificial sweeteners previously listed and mixtures thereof. The amount of artificial sweetener used in the formulation may vary to provide an appropriate amount of sweetness as determinable by one skilled in the art. Mixtures of sweetening and/or flavoring agents are preferably used.

Examples of preservatives suitable for use in the suspension include, for example without limitation, one or more alkyl hydroxybenzoates, such as methyl hydroxybenzoates, ethyl hydroxybenzoates, propyl hydroxybenzoates, butyl hydroxybenzoates and the like. Additional useful preservatives include, but are not limited to, sodium benzoate, potassium sorbate, salts of edetate (also known as salts of ethylenediaminetetraacetic acid, or EDTA, such as disodium edetate) and antimicrobial agents including parabens (p-hydroxybenzoic acids esters) such as methyl paraben, ethylparaben, propylparaben, butylparaben and the like, and combinations thereof. Parabens are preferred, with methyl paraben most preferred for use as preservative ingredients to add to the present formulation, although other pharmaceutically acceptable preservatives may be substituted therefore. Preservative(s) as used in the formulation are present in a pharmaceutically acceptable range.

Examples of suspending/viscosity agents suitable for use in the suspension include but are not limited to gums; sorbitol; glycerol; polyvinyl alcohol; polyvinyl pyrrolidone; polyethylene oxide; cellulose derivatives, such as hydroxypropylmethylcellulose or a salt thereof, alkyl ether of cellulose, such as methylcellulose, ethylcellulose, hydroxyethylcellulose, hydroxyethylmethylcellose and mixtures thereof. Preferably the viscosity-enhancing agent is hydroxypropylmethylcellulose e.g. (HPMC K4M, HPMC K100 LVP; HPMC K15 MP; HPMC E4 MP; HPMC E10 MP CR). The suspension may also contain a dispersing agent(s), which includes but is not limited to, colloidal silicon dioxide and surfactants, wherein the surfactant is used alone or as an admixture with one or more surfactant. Combinations of colloidal silicon dioxide with one or more surfactants can also be used alone or with other pharmaceutically acceptable dispersing agents.

The suspension may also contain a pH stabilizing agent to maintain a desired pH. The term “pH stabilizing agent” encompasses buffers. Suitable pH stabilizing agents include but are not limited to tribasic sodium phosphate, anhydrous sodium carbonate, glycine, citric acid or mixtures thereof.

Preferably, the pH of the suspension formulation is in the range of about 2.0 about 8.0. More preferably, the pH of the suspension formulation is in range from about 4.5 to about 6.0.

The suspension may also contain wetting agent(s) which include, but are not limited to, sorbitan monolaurate, polysorbate 80, and sodium lauryl sulfate and the like. The formulation may also contain suitable coloring agent(s) to provide an appealing color to the suspension, which include, but are not limited to, titanium dioxide pigments, lake colors and iron oxide pigments.

The suspension may also contain suitable antifoaming agents, which include, but are not limited to a simethicone emulsion, dimethicone, lutrol and the like.

The suspension may also contain antioxidant(s) which include, but are not limited to, tocopherols, gallic acid and gallates, butylated hydroxy anisole, butylated hydroxy toluene, ascorbic acid, maleic acid, sodium bisulphate, sodium metabisulphite, sodium formaldehyde sulphoxylate and the like.

In an embodiment, the fixed dose combination formulation in the form of an oral suspension comprising rifaximin and lactulose is prepared by a process comprising the steps of mixing rifaximin and lactulose with one or more additive(s) selected from the group comprising stabilizers, wetting agents, sweeteners, thickening agents, dispersing agents, pH stabilizing agents, flavoring agents, preservatives, coloring agents, and the like. Further, the suspension is prepared by mixing with suspension vehicles well known to persons skilled in the art, such as xylitol, propylene glycol, glycerin, sorbitol, liquid glucose and the like, in addition to water, and then additional milling of the above suspension.

In an embodiment, the fixed dose formulation of the invention is in the form of a sachet, filled with a plurality of separate granules or a powder comprising rifaximin and lactulose. The granules or powder may comprise a mixture of rifaximin and lactulose. The granules can be prepared using a conventional wet granulation, dry granulation, direct compression, or slugging method. The two granulates are mixed and either compressed into tablets or filled into sachets. The rifaximin granulate may be enteric-coated, coated with a protective coating, or coated with a controlled release coating if modified release formulation is desired.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediamine tetraacetic acid; buffers such as acetates, citrates or phosphates and agents for the adjustment of tonicity such as sodium chloride or dextrose. The pH can be adjusted with acids or bases, such as hydrochloric acid or sodium hydroxide.

The injection can be a bolus or by continuous infusion. Depending upon the route of administration, rifaximin can be coated with or disposed in a selected material to protect it from natural conditions that may detrimentally affect its ability to perform its intended function.

Formulations suitable for injectable use include liposomal suspensions, microsphere suspension, sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). The formulation preferably is sterile and should be fluid to the extent that it can be easily pulled into a syringe and then expelled from the syringe. The formulations suitably should be stable under the conditions of manufacture and storage, and preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, or sodium chloride in the formulation. Prolonged absorption of the injectable formulation can be brought about by including in the formulation an agent which delays absorption, for example, aluminum monostearate and gelatin. The injectable preparation can be enclosed in ampoules, disposable syringes or multiple dose vials made of glass or plastic.

For administration by inhalation, the active agents are delivered in the form of an aerosol spray from a pressurized container or dispenser that contains a suitable propellant, e.g., a gas such as carbon dioxide, or a nebulizer. Alternatively, the formulations of the present invention may also be formulated into suitable formulation for pulmonary delivery via dry powder inhalers.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration detergents, bile salts, and fusidic acid derivatives. Transmucosal administration can be accomplished through the use of nasal sprays or suppositories. For transdermal administration, the active agents are formulated into ointments, salves, gels, creams, transdermal patches as generally known in the art.

The formulation can also be prepared in the form of suppositories (e.g., with conventional suppository bases such as cocoa butter and other glycerides) or retention enemas for rectal delivery.

As will be readily apparent to one skilled in the art, the useful in vivo dosage to be administered and the particular mode of administration will vary depending upon the age, weight and mammalian species treated, the particular active agents employed, and the specific use for which these compounds are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved.

Embodiments also provide fixed dose combination formulations comprising rifaximin or a pharmaceutically acceptable salt thereof, lactulose along with a pharmaceutically acceptable carrier. Doses may be selected, for example, on the basis of desired amounts of systemic adsorption, elimination half-life, serum concentration and the like.

Embodiments also provide duration of therapy using the fixed dose combination formulation of the invention. In an embodiment, the fixed dose combination formulation comprising rifaximin or a pharmaceutically acceptable salt thereof and lactulose is administered for greater than 365 days, greater than 730 days, greater than 1095 days, or for the remainder of the life of the patient having HE.

Embodiments also provide frequency of administration of the fixed dose combination formulation of the invention. The fixed dose combination formulation comprising rifaximin or a pharmaceutically acceptable salt thereof and lactulose may be administered once daily, twice daily, thrice daily or multiple times in a day based on patient's requirement for effective management of HE.

Another embodiment includes articles of manufacture that comprise, for example, a container holding a formulation suitable for oral administration of the fixed dose combination of rifaximin and lactulose along with printed labeling instructions providing a discussion of when a particular dosage form extends remission of HE or prevents or delays future episodes of HE. The dosage can be modified for administration to a patient suffering from HE, or include labeling for administration to a patient suffering from HE. The formulation will be contained in any suitable container capable of holding and dispensing the dosage form and will further be in physical relation with the appropriate labeling. The labeling instructions may be consistent with the methods of treatment as described hereinbefore. The labeling may be associated with the container by any means that maintain a physical proximity of the two, by way of non-limiting example, they may both be contained in a packaging material such as a box or plastic shrink wrap or may be associated with the instructions being bonded to the container such as with glue that does not obscure the labeling instructions or other bonding or holding means.

### EXAMPLES

An orally administered rifaximin suspension/lactulose solution is described below:

| Ingredient | % w/w |
|---|---|
| Rifaximin | 1-10 |
| Lactulose | 1-30 |
| Flavouring agent | 0.5-3 |
| Sweetening agent (e.g., sucrose) | 10-40 |
| Dispersing agent (e.g., surfactant, colloidal silicone | 0.1-0.5 |
| dioxide) | |
| Buffering agent | 0.1-0.5 |
| Preservative (e.g., methyl paraben, propyl paraben) | 0.1-0.5 |
| Suspending/viscosity enhancing agent (e.g., sorbitol | 5-25 |
| solution, propylene glycol, HPMC) | |
| pH Adjusting agent (e.g., citric acid, glycine) | 0.1-0.5 |
| Taste enhancing agent | 1-5 |
| Purified water | Up to 100 |

The above ingredients can be formulated using conventional formulation techniques to form a fixed dose combination liquid oral dosage form that includes lactulose in solution and rifaximin in suspension. The pH of the liquid dosage form should be between about 2.0 and 8.0, preferably in the range of 4.0 to 6.0, more preferably in the range of 4.5 to 5.0, and most preferably in the range of about 4.7 to about 4.9, a value that is believed to encompass a range having optimal taste masking properties.

The fixed dosage combination formulation should use a ratio of rifaximin to lactulose of 550 mg rifaximin to 20 to 30 grams of lactulose. The orally administered liquid dosage form of the invention should be bioequivalent to oral administration of a tablet of 550 mg of rifaximin followed by oral administration of 20-30 mg of lactulose.

What is claimed is:

1. An oral formulation comprising fixed dose combination of rifaximin or a pharmaceutically acceptable salt thereof and lactulose for preventing, treating, or maintaining remission of hepatic encephalopathy.

2. The oral formulation of claim 1, wherein the daily dose of rifaximin or a pharmaceutically acceptable salt thereof is about 25 mg to about 6000 mg.

3. The oral formulation of claim 1, wherein the daily dose of lactulose or a pharmaceutically acceptable salt thereof is about 10 mg to about 20 gm.

4. The oral formulation of claim 1, wherein the dose of rifaximin or a pharmaceutically acceptable salt thereof is greater than or equal to the dose of lactulose.

5. The oral formulation of claim 1, wherein the combined weight of rifaximin and lactulose is less than about 80% of the weight of rifaximin or lactulose alone required to achieve same therapeutic effect in patient to which the formulation is administered.

6. The oral formulation of claim 1, wherein the ratio of amount of rifaximin or a pharmaceutically acceptable salt thereof to lactulose ranges from about 0.05:1 to about 50:1.

7. The oral formulation of claim 1, wherein the ratio of amount of rifaximin or a pharmaceutically acceptable salt thereof to lactulose ranges from about 1:1 to about 50:1.

8. The oral formulation of claim 1, wherein the formulation comprises from about 30% w/w to about 70% w/w of rifaximin or a pharmaceutically acceptable salt thereof.

9. The oral formulation of claim 1, wherein the formulation comprises from about 20% w/w to about 80% w/w of lactulose.

10. The oral formulation of claim 1, wherein the formulation is in the form of an oral suspension.

11. The oral formulation of claim 1, wherein the formulation is in the form of a sachet.

12. The oral formulation of claim 1, wherein the formulation is in the form of a tablet.

13. The oral formulation of claim 1, wherein the formulation is in the form of a capsule.

14. An oral formulation comprising fixed dose combination of rifaximin or a pharmaceutically acceptable salt thereof and lactulose for treating, preventing or maintaining remission of hepatic encephalopathy, wherein once-a-day administration of said suspension provides steady state blood levels of rifaximin or lactulose that are comparable to steady state blood levels of rifaximin or lactulose achieved with simultaneous administration of separate dosage forms containing the same amount of rifaximin and lactulose.

15. A method of treating, preventing or maintaining remission of hepatic encephalopathy in a patient in need thereof comprising administering the oral formulation of claim 1 to such patient.

16. The method of claim 15, wherein said method comprises administration of said formulation for greater than one or more of 365 days, 730 days, or 1095 days.

17. A method of reducing the frequency of hospitalization visit by a patient suffering from hepatic encephalopathy, comprising administering the oral formulation of claim 1 to said patient.

18. A method of reducing the recurrence of hepatic encephalopathy episodes in a patient in need thereof comprising administering the oral formulation of claim 1 to said patient.

19. A method of reducing the recurrence of hepatic encephalopathy episodes in a patient refractory or non-responsive to rifaximin or lactulose monotherapy comprising administering the oral formulation of claim 1 to said patient.

20. A method of reducing at least one side effect of rifaximin and/or lactulose therapy in patient suffering from hepatic encephalopathy, said side effects being selected from peripheral edema, nausea, dizziness, fatigue, ascites, dehydration, electrolyte disturbances, abdominal cramping, and combinations of said side effects, comprising administering the oral formulation of claim 1 to said patient.
